(19) United States
(12) Patent Application Publication
(10) Pub. No.: US 2008/0253941 A1
(43) Pub. Date: Oct. 16, 2008

(54) ULTRAVIOLET (UV) RADIATION SOURCE-BASED SURFACE DISINFECTION SYSTEM

(76) Inventors: Donald W. Wichers, Albuquerque, NM (US); Anthony Michael Baca, Albuquerque, NM (US); Luis M. Ortiz, Albuquerque, NM (US)

Correspondence Address: Luis M. Ortiz, P.O. Box 4484, Albuquerque, NM, 87196-4484, US

(21) Appl. No.: 11/967,194

(22) Filed: Dec. 29, 2007

Related U.S. Application Data
(60) Provisional application No. 60/877,912, filed on Dec. 29, 2006.

(51) Int. Cl. B01J 19/12

(52) U.S. Cl. 422/186.3

(57) ABSTRACT

A UV radiation-based surface disinfection system disinfects contaminated surfaces by providing disinfecting UV radiation that eliminates pathogens or microbes from the target surface that it irradiates/illuminates. The system includes a housing adapted to contain UV radiation sources and operate as a UV shield and director, at least one UV radiation (light) emitting source mounted within said housing for providing disinfecting UV radiation from said housing onto a target surface, and at least one of a range sensor and timer. UV radiation source(s) can be mounted on a rotating or sliding assembly within the main assembly to disinfect the preferred surface. The UV radiation source(s) can also be mounted in a portable handheld assembly having a range sensor for enhanced safety during use. The system can be powered by standard AC electrical power or batter sources.

### FILING PRIORITY

The present invention claims priority to U.S. Provisional Patent Application 60/877,912, filed Dec. 29, 2006 by Donald W. Wichers et al, entitled “Ultraviolet (UV) Light-Based Surface Disinfection System and Method.”

### TECHNICAL FIELD

Embodiments are generally related to a surface disinfectant system. Embodiments are also related to surface disinfectant system using Ultraviolet (UV) light sources. Embodiments are additionally related to UV light-based surface disinfectant system incorporated with housing including at least one of a range sensor, a timer, rotator and lens.

### BACKGROUND OF THE INVENTION

Sterilization of surfaces and equipment is becoming more important as disease more readily and easily spreads from contaminated surfaces. Salmonella and staphylococcus-aureus (Staph) are two bacteria that can be easily spread on common surfaces. Staph infection in particular has become a growing concern because it can result in death and is becoming resistant to treatment by drug therapy. The number of persons who have serious, and often termed “invasive,” infections with methicillin-resistant Staphylococcus-aureus (MRSA) in the U.S. is much greater than originally estimated, according to a study reported in the Oct. 17, 2007 issue of the Journal of the American Medical Association. Scientists have found that in about 27 percent of the cases, persons got the infections while patients in hospitals. About 14 percent got the infection in the community. And about 58 percent got the infection after a healthcare encounter, such as hospitalization or surgical procedure. An increase in infection is being reported. More effective, less complicated methods and system for wider sterilization is apparently needed more than ever at this time.

Many processes and devices have been used for cleaning and disinfecting surfaces. Many solutions involve the use of harmful chemicals or extensive labor. It is believed by the present inventors that effectiveness and simplicity will popularize the use of UV systems to aide in the disinfection and cleaning of surfaces found within homes and work environments. There are no hazardous chemicals to work with and UV light sources can kill on site with very impressive results.

The term ultraviolet or UV light, as it is commonly referred to is a proven means of addressing microbiologically contaminated surfaces. This simple, safe surface technology is suitable for both small residential applications as well as large commercial projects. Disinfection in its literal sense means free from infection. As per the U.S. Environmental Protection Agency (EPA) and World Health Organization, sterilization implies disinfection of all forms of life. For practical purpose the term sterilizer is used herein as a generic term to describe UV technology.

The UV spectrum is divided into four regions designated as Vacuum UV, UV-A, UV-B, and UV-C. The UV-A or long-wave UV, which occurs between 325-390 nm bands, is represented by naturally occurring sunlight. This range has little germicidal value. UV-B or middle-wave UV occurs between 295-325 nm and is best known for its best use in sun tanning lamps. These middle-waves may also be found in sunlight and provide some germicidal effect if exposure is sufficient. UV-C or short UV occurs between 200-295 nm and is where the most effective germicidal action occurs. The optimal UV germicidal action occurs at or around 250-265 nm.

When exposed to sunlight, germs are killed and bacteria and fungi are prevented from spreading. Using technology, this natural disinfection process can be utilized most effectively by applying UV radiation in a controlled way. Surface disinfection makes most sense when the targeted surfaces have minimum resistance to microbic contamination. When applied, short wave UV light penetrates the cell structure of the micro-organism and destroys the DNA structure thus preventing it from replicating. Due to UV-C light's rapid action it can be used on moving as well as static surfaces. UV radiation is an effective and secure way to disinfect surfaces and the system is easy to operate and maintain, which brings down total running costs. UV radiation provides the most effective means for maintaining a bacteria free environment.

In order to address a need for improved surface disinfection solutions, the present inventors provide disclosure herein of an improved UV radiation system for surface disinfection.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the present invention to provide for an improved UV light source-based disinfection apparatus for disinfecting target surfaces using UV radiation. UV light can be provided in the form of UV lamps, UV semiconductor lasers, UV diodes and/or UV light emitting diodes (LEDs). UV radiation is preferably provided in the UV-C radiation frequency range.

It is another aspect of the present invention to provide for a UV light emitting source for a disinfecting illumination including a UV disinfecting system for enabling surface disinfection comprising a plurality of UV light emitting sources mounted within an UV assembly including a housing adapted to operate as a UV shield and director adapted for providing disinfecting UV light from the UV assembly directly onto a target surface area.

It is another aspect of the present invention that a system as described herein can include an optional range sensor incorporated with the UV assembly to detect when the housing and laser source are within safe operating range of a target surface. Use of a range sensor can reduce the amount of UV that is illuminated outside of the housing or away from the target surface.

It is another aspect of the present invention that the system can include a built-in timer customizing start time and run time functions.

It is another aspect of the present invention that a system as described herein can include at least one rotator to mount light emitting sources and provide sweeping movement of radiation from light sources onto target surfaces.

It is another aspect of the present invention that the system can include at least one lens to enhance better focusing of the light.

It is another aspect of the present invention that the system can include a handle extending from the housing to enable portable hand operation of the system by a user and facilitating extension of the UV emitting sources away from the user during operation.

It is another aspect of the present invention that the system can include a built-in DC power source to enable portable use of the system. The power source can be rechargeable.

It is a further aspect of the present invention to provide for a rotator and lenses, incorporated to the main assembly to provide a better coverage and focus for the device's UV radiation output and ultimate contact a target surface to be disinfected.

The aforementioned aspects and other objectives and advantages can now be achieved as described herein. The UV light emitting sources emit radiation onto the surface to be disinfected. UV radiation effectiveness can be improved by using any combination of a housing, rotators and lenses to provide better focus. The light source can preferably be kept to wavelengths with in the deep UV-C region. When light emitting sources are mounted on a rotating or sliding assembly, a target surface can effectively be swept with UV radiation emanating from the UV source(s). The apparatus can be built with a runtime timer or range sensor to enhance safety and customize apparatus function. The apparatus can run on standard electrical energy (120 VAC) or reduced electrical requirements such as provided by battery sources used to power semiconductor UV light sources (e.g., UV LEDs and UV diodes).

### BRIEF DESCRIPTION OF THE DRAWINGS

The accompanying figures, in which like reference numerals refer to identical or functionally similar elements throughout the separate views and which are incorporated in and form a part of the specification, further illustrate the embodiments and, together with the detailed description, serve to explain the embodiments disclosed herein.

FIG. 1 illustrates the block diagram of a UV light surface disinfectant system, which can be implemented in accordance with a preferred embodiment;

FIG. 2 illustrates an arrangement of UV light emitting surface disinfectant device, which can be implemented in accordance with an alternative embodiment; and

FIG. 3 illustrates another arrangement of UV surface disinfectant device with a single light emitting source preferably confined for a small area, in accordance with an alternative embodiment; and

FIG. 4 illustrates another arrangement for a portable UV surface disinfectant device including a handle, surface sensor and built-in power supply.

### DETAILED DESCRIPTION

The particular values and configurations discussed in these non-limiting examples can be varied and are cited merely to illustrate at least one embodiment and are not intended to limit the scope thereof.

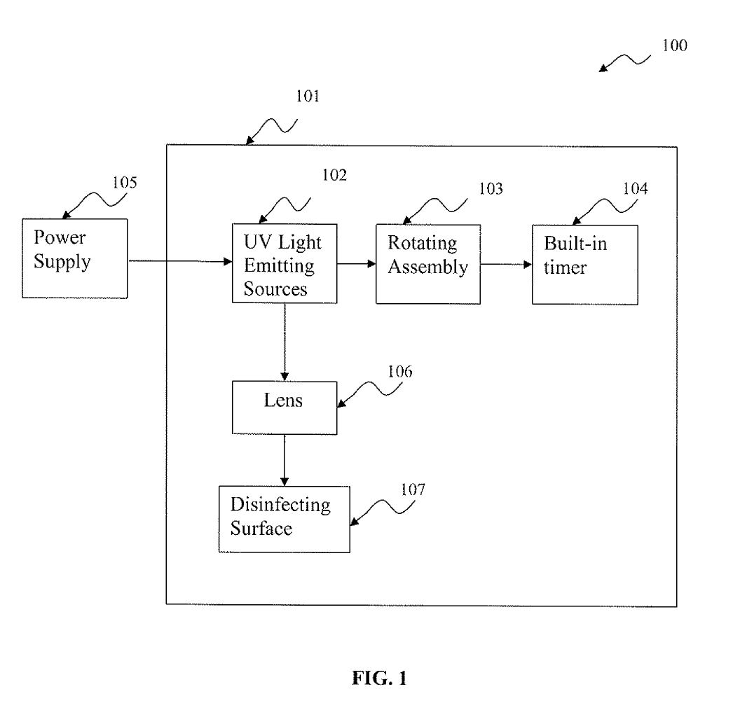

Referring to FIG. 1 a block diagram 100 illustrating the basic components of a UV light-based disinfectant device in accordance with the present invention. One or more UV light emitting sources 102 such as UV lamps, UV semiconductor lasers, UV diodes and/or UV light emitting diodes (LEDs) mounted within a housing 101. These UV radiation sources can be mounted on a stationary, rotating or sliding assembly 103 within the housing 101. A stationary assembly can be effective for handheld, portable applications where a user can sweep a target surface. The UV light source 102 in the system can utilize lenses 104 to render a better focus of radiation onto the desired target surface area. The light source is chosen at wavelengths best suited to kill the microorganisms upon that surface. The apparatus can be built with a built-in timer 106 to allow for customization of particulars such as start times, run times etc. The apparatus includes a power supply 105 enabling it to run off of standard household energy (120 VAC) or reduced energy requirements typically provided by battery sources.

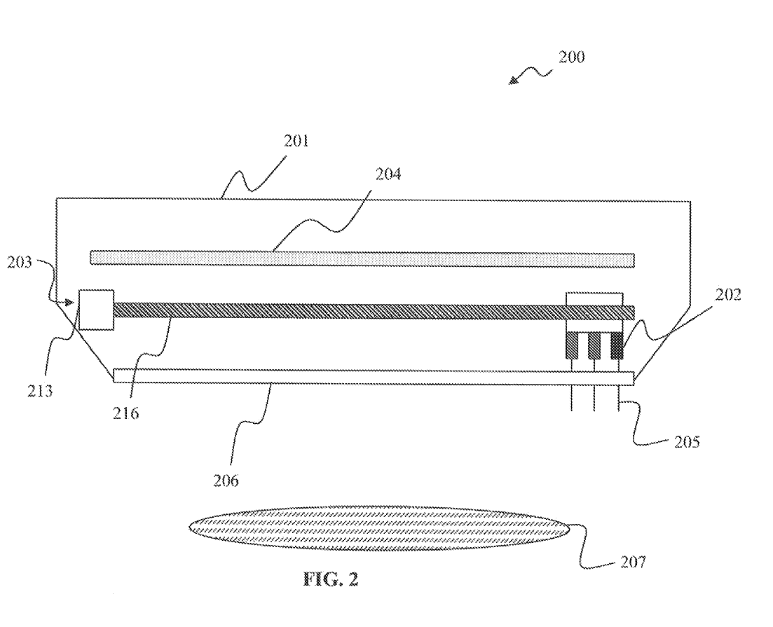

During use, UV radiation from the UV light source can be passed through a lens, when used, and illuminate the target surface 107 to be disinfected. Referring to FIG. 2 shows an arrangement 200 for a UV light disinfectant device 200 including a rotator assembly 203. The whole device is mounted within a housing 201 as illustrated in FIG. 1 which is placed on, near or above a surface to be disinfected. The housing 201 operates as a shield to direct light towards a target source, but minimize the illumination of UV radiation onto a user. The UV light emitting sources 202 as depicted in FIG. 2 preferably can be UV LEDs, UV semiconductor lasers, UV laser diodes, although a single UV lamp can also be utilized, LEDS, diodes and lasers are known to be more focused within the UV-C radiation region and, therefore, can be more effective in sterilization. The UV light emitting sources 102 can be mountable on a rotating assembly 203, as depicted in FIG. 1, within the main assembly so as to effectively sweep the surface. The rotator motor 213 is mounted to the main assembly and is connected to a rotating rod 216. The UV light sources 202 are mounted onto a carrier 217 that is carried by the rotating rod 216 and moves linearly (e.g., slides) along the rotating rod 216 and/or rotates back-forth on the rotating rod 216 to effectively sweep or cover the entire target surface locating underneath the housing 201 and thereby kill the microorganisms upon that surface. Movement of the light sources 202 on the rod are similar to mechanical movement provided by nonanalogous devices such as ink jet printers wherein ink cartridges are moved along a rotating and sliding mechanism. The UV light disinfecting device 200 can also include a reflector 204 which further minimizes the backward propagation of light into the housing and reflects light out of the housing 101 towards to target surface. UV radiation emitted by the UV sources 205 can be most effectively confined to the area or surface 207 by the housing 201 and reflector 204. A lens 206, when or if used, can magnify, spread, or otherwise amplify the effect of UV radiation. The lens, however, must not at as a filter and block UV-C. Materials known to pass UV radiation should be considered for any lens, when used.

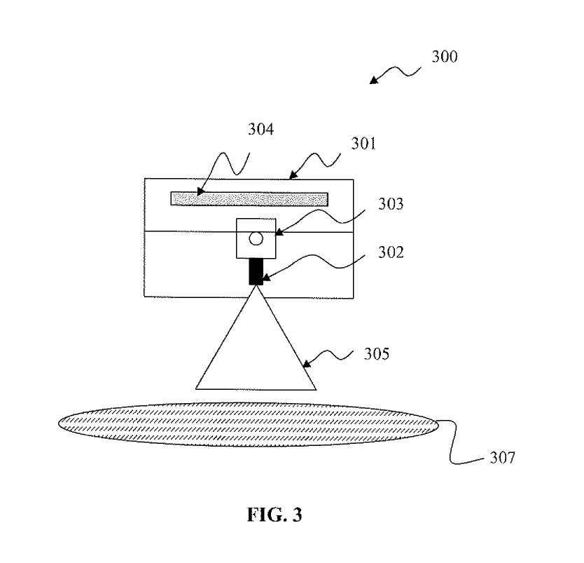

Referring to FIG. 3 is another arrangement of UV disinfectant device 300 illustrated with reference to the present invention. The UV light emitting sources 302 as depicted in FIG. 1 can be mounted within a housing 301. The light emitting source can be mounted on a rotator 303 as depicted in FIG. 2 which can rotate or slide along the main assembly. A shield 304 can be provided to prevent back flow of UV rays into the housing 301. The housing 301 also assists in directing UV radiation towards the disinfecting surface 307 and away from non-targeted areas (e.g., users). The pattern of reflection of UV rays 305 is shown on the disinfecting surface 307.

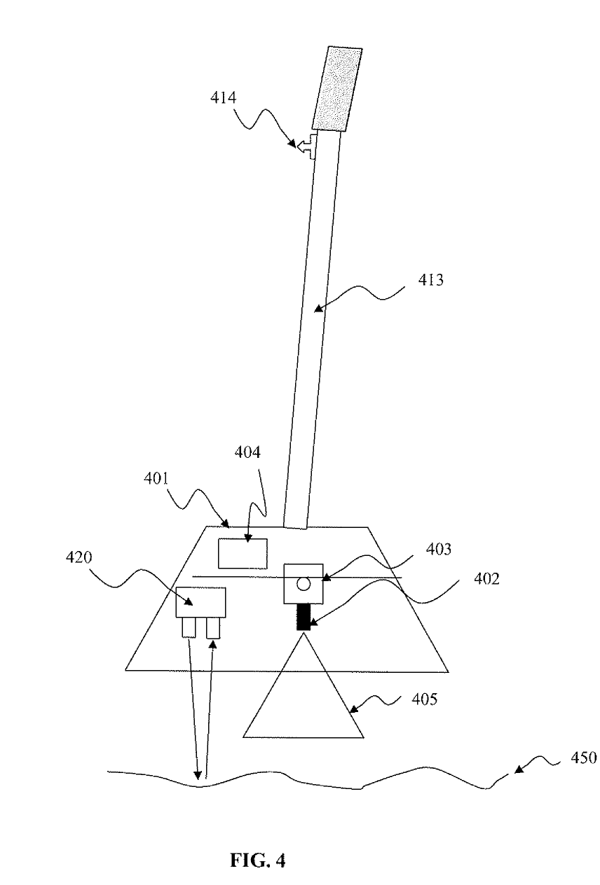

Referring to FIG. 4 another arrangement of UV disinfectant device 400 is illustrated with reference to the present invention. A UV light emitting source 402 is mounted within a housing assembly 401. A power source can provide power to the UV light emitting source 402. UV light emitting source 402 can be optionally mounted on a rotator assembly 403, although portability enables a user to ensure coverage which makes use a rotator optional and, perhaps, superfluous. The housing assembly operates as the UV shield to prevent back flow of UV radiation and emanation of radiation towards and onto a user operating the device 402 near the handle 413 and switch 414. The shield enables more focused/direct application of UV treatment to contaminated surfaces 450. A pattern of illumination of UV rays 405 on the disinfecting surface 450 is shown in FIG. 4. The handle 413 enables an operator/use in handling the system and directing the UV light toward an appropriate target (450). Switch 414 can be integrated into the handle 413 to enable the distal application of power to the UV source 402 by the user. Also, a built-in power source (e.g., batteries) can be incorporated into the housing assembly 401 to enable more portable use of the system by a user. Finally, an optional range sensor 420 can be incorporated within the housing 401 to detect when the housing 401 and laser source 402 are within a safe operational range of a target surface 450. Use of a range finder can reduce the amount of UV that is illuminated outside of the housing and away from the target 450.

The amount of disinfection can depend on a variety of factors including the desired lose of UV light that will achieve the level of disinfection needed for the particular surface, the intensity of the UV light source; and the distance from the UV light source to the surface. The UV light surface disinfection system which includes a UV light source that provides a disinfecting illumination on the specified area or any object surface. A conveyor can be utilized to receive the surface and to rotate or move the surface into the proximity of the UV light source. The conveyor has a roller fixed to it which rotates the UV light source in relation with the disinfecting surface. Lenses can be used to focus or elongate the light into a beam or a spot upon the surface, it must be able to pass UV-C radiation necessary for sterilization. The light source is preferably set to wavelengths in the deep UV-C region at or near 254-277 nm. As the apparatus is supplied with electrical power, the light emitting sources start giving out the rays and the surface is thus disinfected.

Shortwave UV light penetrates the cell-structure of the microorganism and destroys the DNA structure thus preventing it from replicating. Due to UV-C light's rapid action it can be used on moving as well as static surfaces. UV is an effective and secure way of disinfecting surfaces and the system is easy to operate and maintain, which brings down total running costs. UV disinfection equipment is user-friendly and requires less space than other methods. UV simply assures a bacteria free environment. Multiple assemblies can be used to disinfect large area or to decrease the time required to disinfect the area. The UV light emitting devices may be hard mounted to a circuit board, designed to cantilever, etc to affect the best cleaning of the surface.

It will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. An UV disinfecting system for surface disinfection comprising:
a housing adapted to contain UV radiation sources and operate as a UV shield and director;
at least one UV light emitting source mounted within said housing for providing disinfecting UV light from said housing onto a target surface; and
at least one of a range sensor and timer.

2. A system of claim 1, further comprising a rotator assembly to mount the at least one light emitting source, wherein the rotator assembly is adapted to move the at least one light emitting source about a target surface area.

3. The system of claim 1, said at least one UV light emitting source comprising at least one UV LED mounted on a rotator assembly wherein said rotator assembly is further mounted within the main assembly and is adapted to effectively sweep or cover said entire surface to be disinfected with said at least one UV LED.

4. The system of claim 1, said at least one UV light emitting source comprising at least one UV diode mounted on a rotator assembly wherein said rotator assembly is further mounted within the main assembly and is adapted to effectively sweep or cover said entire surface to be disinfected with said at least one UV LED.

5. The system of claim 1, The system of claim 1, said at least one UV light emitting source comprising at least one UV laser mounted on a rotator assembly wherein said rotator assembly is further mounted within the main assembly and is adapted to effectively sweep or cover said entire surface to be disinfected with said at least one UV LED.

6. The system of claim 1, wherein said apparatus can be built with a built-in timer to allow for customization of items such as start times, run times etc.

7. The system of claim 1, wherein the said light source is set to wavelengths in the deep UV-C region at or near 254-277 nm.

8. The system of claim 7, said at least one UV light emitting source comprising at least one of a UV LED, UV diode, UV laser.

9. The system of claim 8, wherein said at least one UV light emitting source is mounted on a rotator assembly wherein said rotator assembly is further mounted within the main assembly and is adapted to effectively sweep or cover said entire surface to be disinfected with said at least one UV light emitting source.

10. An UV disinfecting system for surface disinfection comprising:
a housing adapted to contain UV radiation sources and operate as a UV shield and director;
at least one UV light emitting source mounted within said housing for providing disinfecting UV light from said housing onto a target surface; and
a range sensor adapted to enable operation of said at least one UV light emitting source only when said range sensor is within a specified operating range of the target surface.

11. The UV disinfecting of claim 10 further comprising a rotating assembly adapted as a mount for said UV light emitting sources by a rotator to effectively sweep or cover said entire target surface.

12. The UV disinfecting of claim 10, further comprising a timer adapted for setting a operational runtime for the system.

13. The UV disinfecting of claim 11 further comprising a rotating assembly adapted as a mount for said UV light emitting sources by a rotator to effectively sweep or cover said entire target surface.

14. The UV disinfecting of claim 12, further comprising a timer adapted for setting a operational runtime for the system.

15. The UV disinfecting of claim 10, further comprising a lens mounted within the housing between the at least one UV light emitting source and a target surface.

16. A UV radiation-based system for surface disinfection, comprising:
a housing adapted to contain UV radiation sources and operate as a UV shield and director;
at least one UV light emitting source mounted within said housing for providing disinfecting UV light from said housing onto a target surface; and
a range sensor adapted to enable operation of said at least one UV light emitting source only when said range sensor is within a specified operating range of the target surface; and
a handle extending from the housing to enable portable hand operation of the system by a user.

17. The UV radiation-based system of claim 16 further comprising a switch integrated in the handle and adapted to enable a user to apply electrical power to the at least one UV light emitting source.

18. The UV radiation-based system of claim 16 further comprising a built-in DC power source to enable portable use of the system.

19. The UV radiation-based system of claim 16 wherein said UV light emitting source further comprises at least one UV LED, UV diodes UV laser.

20. The UV radiation-based system of claim 16 further comprising a reflect formed within said housing adapted to reflect UV from within the housing towards a target surface.
